(19) United States
(12) Patent Application Publication
(10) Pub. No.: US 2022/0105227 A1
(43) Pub. Date: Apr. 7, 2022

(54) DUAL CHAMBER ULTRA-VIOLET LED DEVICE FOR USE WITH FACE MASKS TO DISINFECT END-USER'S INHALED AND EXHALED AIR

(71) Applicant: Dynamis Energy, LLC, Eagle, ID (US)

(72) Inventors: Richard C. Newbold, III, Philadelphia, PA (US); Robert W. Abbott, Boise, ID (US)

(21) Appl. No.: 17/490,284

(22) Filed: Sep. 30, 2021

Related U.S. Application Data
(60) Provisional application No. 63/087,057, filed on Oct. 2, 2020.

(51) Int. Cl. A61L 9/20; A62B 18/08

(52) U.S. Cl. CPC A61L 9/20; A62B 18/10; A61L 2209/12; A62B 18/08

(57) ABSTRACT

A breathing protective device including a breathing hose manifold, a dual-chamber sterilization portion including at least one UVC LED lamp, and an end cap. Further disclosed embodiments include, a divider separating the dual-chamber sterilization portion into an intake chamber and an exhaust chamber, an intake check valve in fluid communication with the intake chamber and an inhale port on the breathing hose manifold, an exhaust check valve in fluid communication with the exhaust chamber and an exhale port on the breathing hose manifold, at least one UVC LED lamp in the intake chamber, and at least one UVC LED lamp in the exhaust chamber.

### CROSS-REFERENCE TO RELATED APPLICATIONS

This application, under 35 U.S.C. § 119, claims the benefit of U.S. Provisional Patent Application Ser. No. 63/087,057 filed on Oct. 2, 2020, and entitled “DUAL CHAMBER ULTRA-VIOLET LED DEVICE FOR USE WITH FACE MASKS TO DISINFECT END-USER'S INHALED AND EXHALED AIR,” the contents of which are hereby incorporated by reference herein.

### FIELD OF THE DISCLOSURE

This disclosure relates generally to apparatus and methods for enhancing personal and societal protection from airborne pathogens via air purification chambers. More particularly, this disclosure relates to chambered breathing apparatus that exploits ultra-violet C wavelength (UVC) radiation for its germicidal and disinfectant effects.

### BACKGROUND

Airborne pathogens, such as the COVID-19, SARS, and the like viruses, can cause global pandemics and wreak havoc with societal and economic norms. As airborne pathogens transmit and travel through the air, protective equipment, such as face masks, are often prescribed to mitigate pathogen transmission. However, existing face masks and the like, typically operate to block transmission of airborne pathogens and do not otherwise disinfect inhaled or exhaled air. Blocking transmission is rarely 100% effective and transmission of pathogens can still occur.

In highly infectious environments, such as hospitals or other health care facilities, physicians, nurses, first responders, and other health care workers, can be exposed to unsafe levels of airborne pathogens. In such environments the need for effective protective equipment is enhanced. Other needs for effective protective equipment, and drawbacks of existing solutions, also exist.

### SUMMARY

Accordingly, disclosed embodiments address the above, and other, needs and drawbacks of existing systems and methods. Disclosed embodiments include a breathing protective device including a breathing hose manifold, a dual-chamber sterilization portion including at least one UVC LED lamp, and an end cap. Further disclosed embodiments include, a divider separating the dual-chamber sterilization portion into an intake chamber and an exhaust chamber, an intake check valve in fluid communication with the intake chamber and an inhale port on the breathing hose manifold, an exhaust check valve in fluid communication with the exhaust chamber and an exhale port on the breathing hose manifold, at least one UVC LED lamp in the intake chamber, and at least one UVC LED lamp in the exhaust chamber.

In further disclosed embodiments, the breathing protective device may include an intake port on the end cap in fluid communication with the intake chamber, and an exhaust port on the end cap in fluid communication with the exhaust chamber.

In further disclosed embodiments, the breathing protective device may include a reflective surface on an interior wall of the intake chamber and/or exhaust chamber. In still further disclosed embodiments, the reflective surface comprises a sintered fluoropolymer material. In still further disclosed embodiments, the reflective surface comprises a sintered fluoropolymer material impregnated with barium sulfate.

In further disclosed embodiments, the breathing protective device may include an intake breathing tube in fluid communication with the inhale port on the breathing hose manifold, an exhaust breathing tube in fluid communication with the exhale port on the breathing hose manifold, and a facemask in fluid communication with the intake breathing tube and the exhaust breathing tube.

Other embodiments are also disclosed.

### BRIEF DESCRIPTION OF THE DRAWINGS

FIG. 1A is an isometric perspective view of a portion of a dual-chamber, single housing protective device in accordance with disclosed embodiments.

FIG. 1B is a partially transparent isometric perspective view of a portion of a dual-chamber, single housing protective device in accordance with disclosed embodiments.

FIG. 1C is a partially transparent isometric perspective view of a portion of a dual-chamber, single housing protective device in accordance with disclosed embodiments.

FIG. 2 is a schematic illustration of the protective device as worn on a user in accordance with disclosed embodiments.

FIG. 3 is a partial cut-away isometric view of a portion of a dual-chamber, single housing protective device in accordance with disclosed embodiments.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the spirit and scope of the invention as defined by the appended claims.

### DETAILED DESCRIPTION

Disclosed embodiments include apparatus for enhancing personal and social protection from airborne pathogens via air purification chambers. Embodiments of the device exploit the germicidal efficacy of ultra-violet C wavelength (UVC) radiation supplied by light emitting diodes (LEDs) powered by rechargeable batteries or other power supply. An inhale-exhale embodiment of the protective device provides a dual-chamber configuration with one-way air check valves designed to treat respired air, providing inactivation of pathogens in both inhaled and exhaled air. The volume of each identical dual-chamber is sized to accommodate the average amount of air in one adult breath cycle (e.g. one respiratory tidal volume). Both dual-chambers are housed in one unit, which may be a single cylinder divided into two equal, half-cylindrical spaces each provided with two, one-way/unidirectional check valves to accept/transiently store/disinfect one complete inhalation and one complete exhalation of the end-user.

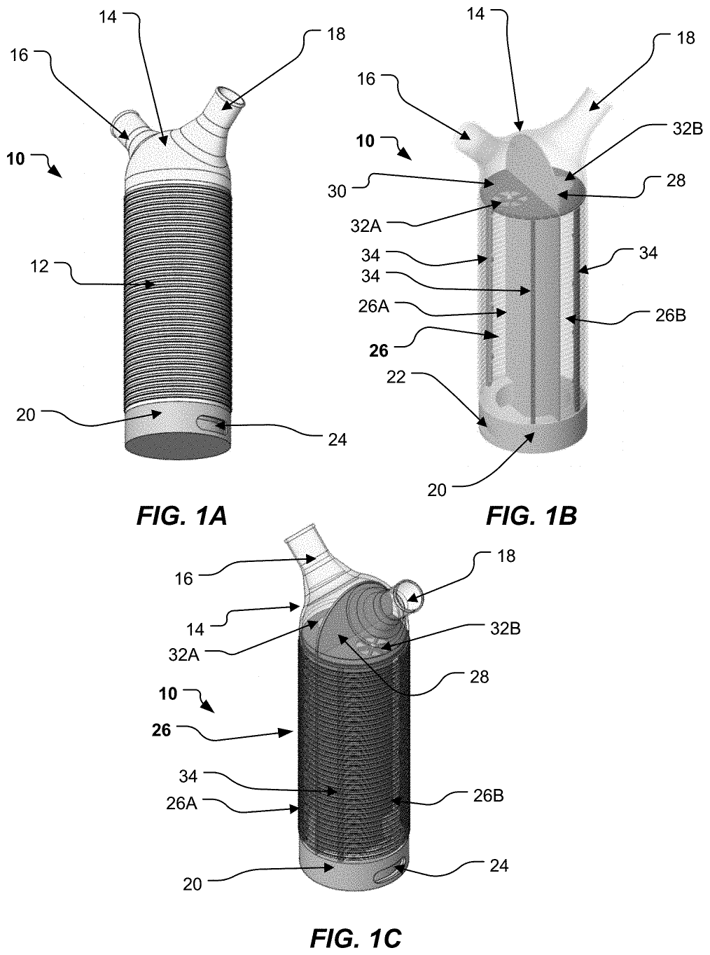

FIG. 1A is an isometric perspective view of a portion of a dual-chamber, single housing protective device 10 in accordance with disclosed embodiments. As shown, protective device 10 includes a housing 12, which is shown as generally cylindrical, but may be other shapes. As also shown, protective device 10 includes a breathing hose manifold 14 with an inhale port 16 and an exhale port 18. As also shown, protective device 10 includes an end cap 20 with an intake port 22 (visible in FIG. 1B) and an exhaust port 24. The shapes and locations for the ports 16, 18, 22, 24 are merely exemplary and those of ordinary skill in the art having the benefit of this disclosure would comprehend that other configurations are possible.

FIG. 1B is a partially transparent isometric perspective view of a portion of a dual-chamber, single housing protective device 10 in accordance with disclosed embodiments. As shown, embodiments of the dual-chamber 26 configuration include an intake/inhale chamber 26A and an exhaust/exhale chamber 26B within housing 12 and separated by a divider 28. One end of the dual chamber 26 includes a check plate 30 that includes an intake/inhale check valve 32A for the intake/inhale chamber 26A and an exhaust/exhale check valve 32B (better viewed in FIG. 1C) for the exhaust/exhale chamber 26B. Inside each chamber 26A, 26B are a number of UVC LED assemblies 34 containing one or more UVC LED lamps 44 (shown in FIG. 3).

FIG. 1C is a partially transparent isometric perspective view of a portion of a dual-chamber 26, single housing protective device 10 in accordance with disclosed embodiments. FIG. 1C better shows the exhaust/exhale chamber 26B side of the device 10.

As those of ordinary skill in the art having the benefit of this disclosure would comprehend, the device 10 could also be deployed as two separate cylinders/chambers of identical size/shape with one dedicated to inhalation and the other to exhalation. Other configurations are also possible.

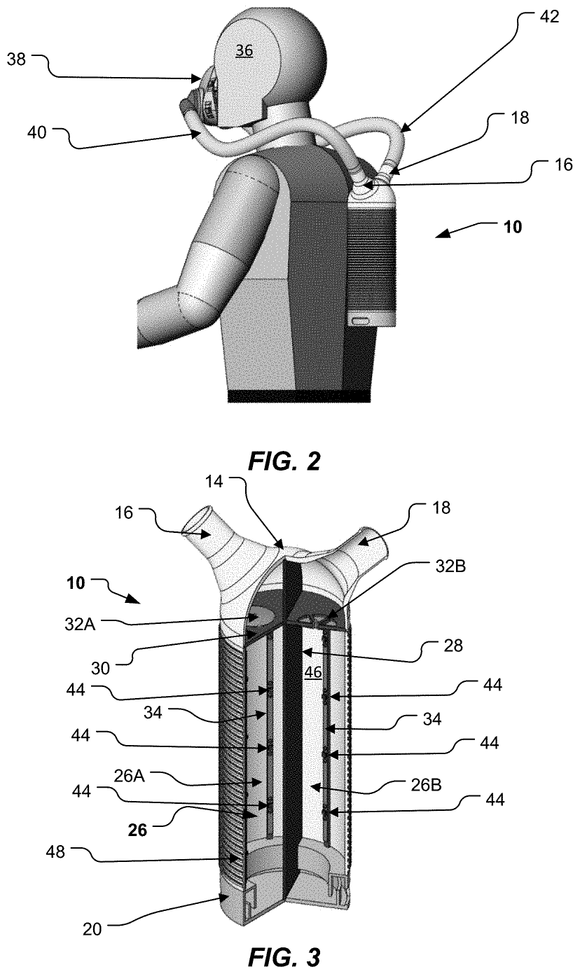

FIG. 2 is a schematic illustration of the protective device 10 as worn on a user 36 in accordance with disclosed embodiments. As shown, user 36 wears a facemask 38 that covers the mouth and nostrils. Facemask 38 may also comprise a full or partial face shield, a helmet, or other protective headgear as would be apparent to those of ordinary skill in the art having the benefit of this disclosure. An intake/inhale breathing tube 40 connects facemask 38 to inhale port 16 and an exhaust/exhale breathing tube 42 connects facemask 38 to exhale port 18. The relative positions of breathing tubes 40, 42 is merely exemplary and they may be swapped to match with the appropriate check valve 32A, 32B. Inspired and expired air flow (inhalation and exhalation) in the dual-chamber 26 configuration may be accomplished solely by human respiration without the use of additional pumps or pressurizers.

FIG. 3 is a partial cut-away isometric view of a portion of a dual-chamber 26, single housing protective device 10 in accordance with disclosed embodiments. As shown, a number of UVC LED lamps 44 may be configured on UVC LED arrays 34. The UVC LED lamps 44 are preferably selected to emit high intensity (e.g., 110 W) UVC light in the germicidal wavelength range of 200 to 280 nm which inactivates airborne pathogenic microbes (bacteria, viruses, fungi and spores).

In some embodiments, a reflective surface 46 is provided on the interior of the dual-chambers 26 (and, in some embodiments, the divider 28) to, among other things, improve the efficiency because UVC photons will continue to reflect in search of pathogenic material rather than be absorbed by a hard surface. In some embodiments, reflective surface 46 provides reflectance (in the range of 90% to 99%), by using sintered fluoropolymers, or the like, for the inner walls of the dual-chambers 26. Exemplary reflective materials are Spectralon by Labsphere, or ODM98 by Gigahertz Optik. The fluoropolymers may also be doped and/or impregnated with barium sulfate which is another material whose characteristic reflectance for UVC light is very high. The reflective surface 46 may be a thin foil with a structural backing, typically aluminum for optimal UVC reflectance, to act as the full wall structure for the dual-chamber 26. Alternatively, the entire wall structure could be a solid casting of suitable sintered fluoropolymers or UVC-transparent fluoropolymers impregnated with barium sulfate or other equally reflecting compound that can be mixed into the resin. These fluoropolymers or other high-reflectance, nonreactive materials are useful because they can have characteristic reflectance in the UVC band of better than 95% and are stable under long-term to germicidal UVC light. Other reflective surfaces 46 are also possible.

As also shown in FIG. 3, embodiments of the housing 12 may include cooling fins 48 to help dispel heat generated by the UVC LED lamps 44. Other configurations are also possible.

In use, inspired and expired air is purified by UVC LED lamps 44 housed in the dual-chambers 26, lined with an ultra-UVC reflecting surface 46, providing purified air for introduction directly into and out of the user's 36 breathing space via one-way check valves 32A, 32B. As discussed, one chamber (e.g., 26A) is dedicated to inhaled air and the other chamber (e.g., 26B) is dedicated to expired air. Both dedicated UVC reflecting chambers 26A, 26B, given their UVC irradiance from the UVC LED lamps 44, the UVC reflective surface 46, and increased residence time, can inactivate virtually all known pathogenic microbes. The face mask 38 (or other protective head gear) and protective device 10 ensures that undiagnosed/unsuspecting/asymptomatic infected users 36 (whether patient, provider, emergency personnel, traveler or other) do not spread microbes into the ambient air around them.

Inspired and expired air enters and exits the protective device 10 via intake 22 and exhaust 24 ports through the result of the ordinary exercise of user 36 breathing. The air passageways are unobstructed with no airway resistance, so no additional respiratory effort is required from the user 36. Pathogens are subjected to high intensity UVC light in the germicidal wavelength range of 200 to 280 nm of sufficient energy to inactivate virtually all airborne pathogenic microbes (bacteria, viruses, fungi and spores) and achieve up to 99.999% sterilization with all known human pathogenic viruses. No pathogenic organisms are resistant to UVC's antimicrobial impact as its mechanism of action is to disrupt DNA and/or RNA reproduction, repair, and translation.

The dual-chambered 26 protective device 10 maximizes the UVC dose delivered to microbes in both chambers 26A, 26B by, at least, increasing the irradiation by incorporating multiple UVC-LED lamps 44, exponentially increasing the irradiation by using a reflective surface 46 to line the chambers 26A, 26B, and maximizing the exposure time in the chambers 26A, 26B by retaining the introduced air (both inhalation and exhalation) with one-way check valves 32A, 32B that hold the air for between 2.5 and 5.0 seconds.

Embodiments may incorporate UVC exposure chambers 26A, 26B of optimized geometry and material properties with the use of one-way air intake and exhale check valves 32A, 32B. Each chamber 26A, 26B consists of UVC LED lamps 44 and a reflective surface 46. The reflective surface 46 may have a reflectance of greater than 90% for UVC germicidal wavelengths. The high reflectance dramatically increases the efficiency of microbial inactivation by UVC photons as compared to polished aluminum or stainless steel. In a high reflectance environment, statistically photons have exponentially more reflection opportunities and thus a greater probability of contact with—and destruction of—pathogenic material at the variable inhalation/exhalation rates required for even intense physical activity.

The user 36 is 100% shielded and protected from exposure to harmful UVC wavelengths which can also damage mammalian cells. Unlike mercury fluorescent UVC bulbs that are still widely used, UVC LED lamps 44 produce no ozone or other toxins and the reflective surface 46 material is 100% inert as well as long-lasting. Embodiments of the compact protective device 10 may be powered by small, long life batteries (e.g., replaceable or rechargeable) which may be located externally to the housing 12, within the breathing hose manifold 14, within the end cap 20, or in some other location, and obviating the need for an external powered air purifying device (PAPR) or heavy battery pack.

The UVC chambers 26A, 26B can have any convenient cross-sectional area, though circular, spherical and rectangular envelopes are the most common. One protective device 10 can be fitted with multiple UVC sterilization chambers 26 and with intake/exhaust valves 32A, 32B. Multiple UVC chambers 26A, 26B can be placed in series to further increase the germicidal efficacy. Additionally, UVC chambers 26A, 26B can be manufactured with larger interior diameters to increase air residence time and exposure to UVC radiation by adding interior chamber volume. UVC chambers 26A, 26B can also be manufactured in different lengths to accept longer UVC LED lamps 44 of higher wattage (currently up to 110 watts), thereby increasing UVC LED irradiance concentration and increasing germicidal efficacy.

In some embodiments, system controls can include monitoring of LED ballasts to determine if any UVC LED lamps 44 may not be functioning. Any UVC LED lamp 44 that is not functioning properly can cause an LED warning light and/or audible alarm to appear/sound on a control panel, or the like, which will alert the user 36 to potentially non-functioning UVC LED lamps 44.

The embodiments disclosed here can be adapted and configured for use with any commercially manufactured facemask 38 of virtually any design. It can also be applied to room filtration, air conditioning or heating units for purposes of air purification.

Although various embodiments have been shown and described, the present disclosure is not so limited and will be understood to include all such modifications and variations are would be apparent to one skilled in the art.

What is claimed is:

1. A breathing protective device comprising:
a breathing hose manifold;
a dual-chamber sterilization portion including at least one UVC LED lamp; and
an end cap.

2. The breathing protective device of claim 1 further comprising:
a divider separating the dual-chamber sterilization portion into an intake chamber and an exhaust chamber;
an intake check valve in fluid communication with the intake chamber and an inhale port on the breathing hose manifold;
an exhaust check valve in fluid communication with the exhaust chamber and an exhale port on the breathing hose manifold;
at least one UVC LED lamp in the intake chamber; and
at least one UVC LED lamp in the exhaust chamber.

3. The breathing protective device of claim 1 further comprising:
an intake port on the end cap in fluid communication with the intake chamber; and
an exhaust port on the end cap in fluid communication with the exhaust chamber.

4. The breathing protective device of claim 2 further comprising:
a reflective surface on an interior wall of the intake chamber.

5. The breathing protective device of claim 4 wherein the reflective surface comprises a sintered fluoropolymer material.

6. The breathing protective device of claim 4 wherein the reflective surface comprises a sintered fluoropolymer material impregnated with barium sulfate.

7. The breathing protective device of claim 2 further comprising:
a reflective surface on an interior wall of the exhaust chamber.

8. The breathing protective device of claim 7 wherein the reflective surface comprises a sintered fluoropolymer material.

9. The breathing protective device of claim 7 wherein the reflective surface comprises a sintered fluoropolymer material impregnated with barium sulfate.

10. The breathing protective device of claim 2 further comprising:
an intake breathing tube in fluid communication with the inhale port on the breathing hose manifold;
an exhaust breathing tube in fluid communication with the exhale port on the breathing hose manifold; and
a facemask in fluid communication with the intake breathing tube and the exhaust breathing tube.
